(19) United States
(12) Patent Application Publication
(10) Pub. No.: US 2011/0028524 A1
(43) Pub. Date: Feb. 3, 2011

(54) PHARMACEUTICAL COMPOSITIONS FOR POORLY SOLUBLE DRUGS

(76) Inventors: Ashish Chatterji, New Brunswick, NJ (US); Zedong Dong, Princeton, NJ (US); Harpreet K. Sandhu, West Orange, NJ (US); Navnit Hargovindas Shah, Clifton, NJ (US)

Correspondence Address: HOFFMANN-LA ROCHE INC.;PATENT LAW DEPARTMENT, 340 KINGSLAND STREET, NUTLEY, NJ, 07110, US

(21) Appl. No.: 12/902,186

(22) Filed: Oct. 12, 2010

Related U.S. Application Data
(63) Continuation of application No. 12/114,844, filed on May 5, 2008.
(60) Provisional application No. 60/928,804, filed on May 11, 2007.

(51) Int. Cl. A61K 31/427

(52) U.S. Cl. 514/371

(57) ABSTRACT

The invention relates to solid dispersions of poorly soluble compounds formed by co-precipitation and hot melt extrusion, resulting in improved stability and bioavailability. The invention also relates to hot melt extrusion processes used to prepare such solid dispersions.

### PRIORITY TO RELATED APPLICATIONS

This application is a continuation of U.S. application Ser. No. 12/114,844, filed May 5, 2008, now pending; which claims the benefit of U.S. Provisional Application No. 60/928,804, filed May 11, 2007. The entire contents of the above-identified applications are hereby incorporated by reference.

### BACKGROUND OF THE INVENTION

With the implementation of high-throughput screening in the pharmaceutical industry, a significant number of poorly water-soluble compounds have been identified. Such poorly water-soluble drugs pose significant hurdles for drug bioavailability that in turn affect in vivo efficacy and safety during all stages of development.

Poorly soluble compounds also present technical difficulties in development. One such difficulty is that poor solubility and dissolution result in lower absorption and reduced bioavailability. Another such difficulty is high inter and intra subject variability in pharmacokinetic properties, requiring a wider safety margin. These compounds often require a high dose to achieve the desired therapeutic effect, resulting in unwanted side effects. Further, such compounds often have potential for food effects on bioavailability that complicate the dosing regimen

Consequently, innovative pharmaceutical technologies are being developed to improve the desired properties of such poorly soluble drugs, including but not limited to the following (See R Liu, Water-Insoluble Drug Formulation, Interpharm Press, 2000): particle size reduction, lipid formulation, cosolvents, complexation, co-crystallization, and solid dispersions.

Due to their poor solubility, the absorption/bioavailability of some compounds is dissolution rate limited. A reduction in particle size improves the dissolution rate significantly, which provides better absorption potential and potentially leads to improved therapeutics. Wet milling (see, e.g., U.S. Pat. No. 5,494,683) and nano-technology (see, e.g., PCT Int. Appl. WO 2004/022100 and U.S. Pat. Nos. 6,811,767; 7,037,528; and 7,078,057) are two examples of techniques that can be applied to poorly water-soluble drugs to reduce particle size.

Poorly soluble drugs may dissolve in a lipid-based vehicle at a much higher concentration than in aqueous media. Therefore, formulating the drugs in a lipid formulation may improve therapeutic characteristics of such drugs. After being dosed, the lipid formulation is dispersed in gastric and intestinal fluid, which provides a large surface area for the drug to diffuse from its solution in the lipid to the gastric or intestinal fluid. The high solubility of the drug in the lipid formulation provides a strong driving force for diffusion. Self-emulsifying drug delivery system (SEDDS) is one example of lipid formulation technology. Depending on the selection of the lipid vehicle, the resulting aqueous dispersion may yield a very fine or a crude emulsion (see, e.g., U.S. Pat. Nos. 5,969,160; 6,057,289; 6,555,558; and 6,638,522).

Cosolvents can be used to formulate poorly water soluble drugs for better solubilization and consequently better bioavailability. (see, e.g., U.S. Pat. No. 6,730,679)

Complexing agents, such as cyclodextrins and their derivatives, can be used to solubilize drugs with poor solubility for parenteral formulation (see, e.g., U.S. Pat. No. 7,034,013) or improved bioavailability for oral formulation (see, e.g., U.S. Pat. No. 6,046,177; M J Habib, Pharmaceutical Solid Dispersion Technology, Technomic Publishing Co., Inc. 2001; and T Loftsson and M E Brewster, J. Pharm. Sci. 85(10): 1017-1025, 1996).

Poorly water-soluble drugs may form co-crystals with other compounds that have improved solubility. Therefore, the co-crystals of these drugs can be used for development to provide improved solubility and bioavailability. (see, e.g., U.S. Patent Application No. 2005-267209)

Solid dispersion is an approach to disperse a poorly soluble drug in a polymer matrix in solid state. The drug can exist in amorphous or microcrystalline form in the mixture, which provides a fast dissolution rate and/or apparent solubility in the gastric and intestinal fluids. (see, e.g., A T M Serajuddin, J. Pharm. Sci. 88(10): 1058-1066 (1999) and M J Habib, Pharmaceutical Solid Dispersion Technology, Technomic Publishing Co., Inc. 2001) Several techniques have been developed to prepare solid dispersions, including co-precipitation (see, e.g., U.S. Pat. Nos. 5,985,326 and 6,350,786), fusion, spray-drying (see, e.g., U.S. Pat. No. 7,008,640), and hot-melt extrusion (see, e.g., U.S. Pat. No. 7,081,255). All these techniques provide a highly dispersed drug molecule in a polymer matrix, usually at the molecular level or in a microcrystalline phase. Solid dispersion systems provide a large surface area of the compounds for the dissolution process, which greatly improves dissolution. Therefore, the absorption of these compounds can be improved, if intestinal permeability is not the limiting factor, i.e. biopharmaceutical classification system (BCS) class 2 compounds (Amidon et al., 1995). The amorphous or the microcrystalline API in solid dispersion is more stable than its pure form in the same physical state due to the interaction between the molecules of the polymer and the active pharmaceutical ingredient (API) molecules in the solid dispersion (Matsumoto and Zografi, 1999). However, the solid dispersions prepared from different methods can differ in properties, such as porosity, surface area, density, stability, hygroscopicity, dissolution and therefore bioavailability.

It is possible that the use of different processes to prepare solid dispersion may result in different physico-chemical properties. For example, co-precipitation and spray drying generally provide more porous network resulting in large surface area. The large surface area has fast dissolution rate and may provide rapid onset of action. However, solid dispersions prepared from hot-melt extrusion are generally denser and tend to exhibit a smaller surface area, which may provide a sustained drug release profile in vivo. In spite of these generalizations there is no evidence in the literature suggesting the superiority of one method over another to achieve the desired pharmacokinetic profile, particularly better dose proportionality.

In a U.S. Pat. No. 6,350,786, solid dispersions using water-insoluble ionic polymers with a molecular weight greater than 80,000 D are disclosed to provide a stable amorphous formulation. U.S. Pat. No. 6,548,555 describes the use of ionic polymers, including hydroxypropylmethyl cellulose acetate succinate (HPMCAS), to prepare solid dispersions for improved solubility and better bioavailability.

Despite the variety of formulation tools available to the pharmaceutical scientist, it may not be possible to satisfactorily tailor the pharmacokinetic profile of such poorly soluble compounds, particularly the dose dependent exposure, which is very important to manage the safety and efficacy of the compound. Some supersaturated formulations, such as systems solubilized by cosolvents or solid dispersion approaches, may revert back to crystalline form, resulting in loss of bioavailability at higher dose.

### SUMMARY OF THE INVENTION

The present invention provides solid dispersions of a poorly soluble drug using a hot melt extrusion process to achieve higher bioavailability and superior dose proportionality. The invention focuses on achieving better control of the pharmacokinetic (PK) profile in addition to improving the bioavailability.

In particular, the present invention provides a solid dispersion formulated using hot melt extrusion of (2S,3S)-2-{(R)-4-[4-(2-hydroxy-ethoxy)-phenyl]-2,5-dioxo-imidazolidin-1-yl}-3-phenyl-N-(4-propionyl-thiazol-2-yl)-butyramide (HEP), the structure of which is depicted in FIG. 1, which has poor solubility in aqueous vehicles. The solid dispersion comprises HEP and HPMC-AS. This solid dispersion exhibits higher bioavailability and superior dose proportionality as compared to solid dispersions containing the same components prepared by co-precipitation.

The present invention also provides a method for preparing a solid dispersion of a poorly soluble drug using hot melt extrusion and co-precipitation.

The present invention provides a solid dispersion comprising a compound having an aqueous solubility of less than 1 mg/ml and an ionic or nonionic polymer.

The solid dispersion according to the invention can comprise a compound having an aqueous solubility of less than 1 mg/ml and an ionic or nonionic polymer, wherein the solid dispersion has a higher bioavailability than the crystalline form of the compound.

The solid dispersion according to the invention can comprise a compound having an aqueous solubility of less than 1 mg/ml and an ionic or nonionic polymer wherein the compound exists in an amorphous form.

### BRIEF DESCRIPTION OF THE DRAWINGS

FIG. 1 shows the molecular structure of (2S,3S)-2-{(R)-4-[4-(2-hydroxy-ethoxy)-phenyl]-2,5-dioxo-imidazolidin-1-yl}-3-phenyl-N-(4-propionyl-thiazol-2-yl)-butyramide (HEP).

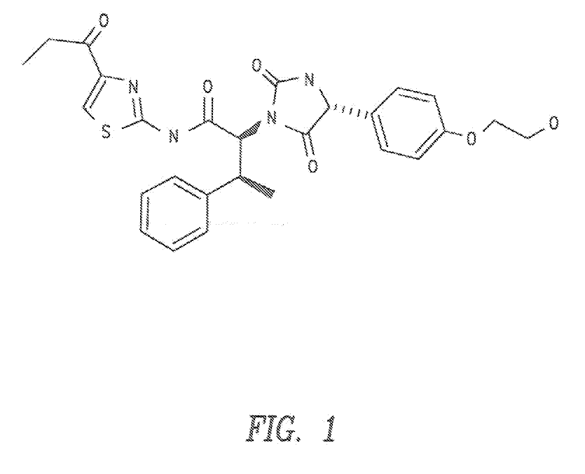

FIG. 2 is a powder X-ray diffraction (PXRD) pattern of the solid dispersion prepared in Example 1, indicating the amorphous nature of the co-precipitate (CP).

FIG. 3 is a powder X-ray diffraction pattern of the solid dispersion prepared in Example 2, indicating the amorphous nature of the hot melt extrudate (HME).

FIG. 4 is the dissolution profiles of the CP and HME products in 1% SLS pH 6.8 50 mM phosphate buffer, prepared in Examples 1 and 2, respectively, showing that the CP product has a faster dissolution rate.

FIG. 5 is the intrinsic dissolution profiles of the CP and HME products in 1% SLS pH 6.8 50 mM phosphate buffer.

FIG. 6 is the water vapor sorption/desorption curve of the CP product, prepared in Example 1.

FIG. 7 is the water vapor sorption/desorption curve of the HME product 2, prepared in Example 2.

FIG. 8 shows the powder X-ray diffraction patterns of the CP product in suspension for a week.

FIG. 9 shows the powder X-ray diffraction patterns of the HME product in suspension for a week.

FIG. 10 shows the powder X-ray diffraction patterns of the CP product in 40° C./75% RH chamber for three months (RH=relative humidity, wherein the relative humidity of an air-water mixture is defined as the ratio of the partial pressure of water vapor in the mixture to the saturated vapor pressure of water at a given temperature).

FIG. 11 shows the powder X-ray diffraction patterns of the HME product in 40° C./75% RH chamber for three months.

### DETAILED DESCRIPTION OF THE INVENTION

The following definitions of the general terms used in the present description apply irrespective of whether the terms in question appear alone or in combination. It must be noted that, as used in the specification and the appended claims, the singular forms “a”, “an,” and “the” include plural forms unless the context clearly dictates otherwise.

As used herein, hot melt extrusion is the process of mixing two or more components using high shear mixing and controlled temperature capability of the extruder. The hot melt extruder consists of four primary parts: motor that controls the rotation of the screws, the screws (primary source of shear and moving the material), the barrels that house the screws and provide temperature control and the die (the exit port) that controls the shape and size of the extrudates. The powder material (either granular or in powder form) is generally fed into the extruder feeding port at controlled rate while the extruder screws are rotating. The material is then conveyed forward using the rotation of screw and the friction of the material against the barrel surface. Depending on the type of extruder, a single screw or a twin screw may be used to operate either in counter or co-rotating mode. The screws can be appropriately designed to achieve required degree of mixing. In general the barrels are segmented to enable the temperature adjustment in each zone throughout the screw length. The exit port (the die system) controls the shape and size of the extrudates.

Co-precipitation is the process of precipitating two or more components together from solution by one of these methods; including, but not limited to, non-solvent addition, temperature change, pH modification or evaporation.

The term “compound having an aqueous solubility of less than 1 mg/ml,” means a compound where the maximum amount of compound that can be dissolved in aqueous fluids (water, simulated gastric and intestinal fluids, aqueous buffers pH 1-8) at 15-30° C. is 1 mg/ml or less.

An ionic polymer is a polymeric excipient with repeat monomeric units that have ionizable groups. The ionic polymers are generally not soluble in water but can be solubilized using pH modification depending on the type of ionizable groups. For example, Eudragit E100® (Degussa) has quarternary ammonium groups that are ionized at pH<5 enabling the solubilization of this particular polymer at low pH's.

A nonionic polymer is a polymeric excipient with repeat monomeric units that do not have any ionizable groups, therefore their solubility is pH independent.

Nonlimiting examples of ionic and nonionic polymers useful in the present invention are polymethylmethacrylates, polyvinylpyrrolidone, hydroxyethyl cellulose, hydroxypropyl cellulose, hydroxypropyl methylcellulose, ethylcellulose, polyvinypyrrolidone-polyvinylalcohol, hydroxypropyl methylcellulose acetate succinate, hydroxypropyl methylcellulose phthalate, polyvinyl acetate phthalate, cellulose acetate phthalate, hydroxypropyl cellulose acetate phthalate, methylcellulose acetate phthalate and polymeric surfactants such as poloxamers. The preferred polymer is hydroxypropyl methylcellulose acetate succinate.

The term “physically stable” as used herein means that no crystallization peaks are detected using X-ray diffraction method after storage in 40° C./75% RH chamber for 12 weeks.

Hypromellose acetate succinate (HPMC-AS) or hydroxypropyl methylcellulose acetate succinate is an enteric coating material for enteric or sustained release formulations. It is also used in solid dispersion technology for poorly water-soluble compounds to improve bioavailability. With various contents of acetyl and succinoyl groups in the polymer, there are several types of HPMC-AS, which dissolve at different pH levels. Type L has a high ratio of succinoyl substitution to acetyl substitution (S/A ratio), while type H has a low S/A ratio and type M has a medium S/A ratio. With a high S/A ratio, type L HPMC-AS dissolves at a lower pH (≧5.5), compared with pH≧6.0 for type M and pH≧6.8 for type H (Shin-Etsu Chemical Co., Ltd.) All of the grades are suitable for preparing the solid dispersions using both methods (HME & CP).

The present invention provides an approach to prepare a solid dispersion of a poorly soluble drug using a hot melt extrusion process to achieve higher bioavailability and superior dose proportionality.

The amorphous form (molecular dispersion) of the drug is desired because it generally has better solubility or dissolution as compared with the crystalline form.

HEP (See PCT Int. Appl. WO 2006/018188 and WO 2006/029862) is a MEK1/2 inhibitor that has poor aqueous solubility. When the crystalline form was dosed in animal species even in the nanocrystalline form, HEP provided a very low exposure. The present invention provides solid dispersions of HEP in amorphous form having improved bioavailability.

Solid dispersions of HEP were prepared as described in the appended examples using co-precipitation, hot-melt extrusion, and spray drying. In each instance, the same ratio of HEP and HPMC-AS were employed.

The amorphous formulations produced by HME and CP were further characterized by several complementary techniques. The drug in both the co-precipitate (CP) and the hot melt extrudate (HME) were in amorphous form as shown by their powder X-ray diffraction (PXRD) patterns. However, the solid dispersion prepared by spray drying did not provide the amorphous form of the drug. The CP and HME products have similar glass transition temperatures at 106° C. and 104° C., respectively. Under polarized microscope, neither of the two products showed any birefringence. The particle morphology of the CP product is flake-like, while the HME product appears as glass-like particles with an irregular shape. SEM micrographs of the two products indicate that the co-precipitation process produced porous particles with rough surfaces, while hot melt extrusion process produced particles with smooth surfaces and sharp edges. According to the BET results, the CP product had a specific surface area of 6.29 m²/g compared with 0.13 m²/g for the HME product, which confirms the surface properties observed in the SEM micrographs. However, the two products have comparable true densities with 1.33 g/cm³ for the CP product and 1.30 g/cm³ for the HME product.

Water vapor sorption/desorption experiments suggested that the two products have similar overall hygroscopicity and no crystallization of HEP occurred in the samples after experiments under microscopic examinations. However, in the adsorption isotherm, the CP product took up slightly more water than the HME product. There was no significant difference in the desorption isotherm between the two products. Considering the much larger specific surface area of the CP product, unexpectedly, there was less moisture per surface square unit. This slight difference between the two products cannot be distinguished by DSC or spectroscopic tools.

However, further in vitro and in vivo evaluation of these formulations provided the differentiation of the products, particularly in terms of the stability and bioavailability.

The dissolution was conducted using the USP paddle method in 500 ml 1% SLS 50 mM phosphate buffer, pH 6.8. The CP product had much faster dissolution than the HME product, apparently due to the difference in specific surface area. It took about half an hour to achieve 100% release for the CP product, compared with eight hours for the HME product. Using the same experimental conditions, the intrinsic dissolution rate (IDR) was determined as 0.040±0.006 mg/minute/cm² and 0.070±0.003 mg/minute/cm² for the CP and HME products, respectively. In addition, after intrinsic dissolution experiments, the pellet surfaces for both products were examined by PXRD and microscopy and the results indicated no crystallization.

Further evaluation of the amorphous form produced by co-precipitation and hot-melt extrusion showed significant improvement in bioavailability of the drug. Although the bioavailability of the two formulations was comparable, the dose dependent exposures were significantly different. The solid dispersion prepared by the hot-melt extrusion process exhibited superior dose dependent exposure when tested in vivo at doses of 50 mg/kg and 250 mg/kg as compared to the solid dispersion prepared by the co-precipitation process at the same doses. This result was unexpected and suggests that the solid dispersion prepared by hot melt extrusion can provide better control of the dose response curve.

In addition, the solid dispersion prepared by hot-melt extrusion has better physical stability in suspension and provides a sustained release profile when compared to the solid dispersion prepared by co-precipitation. As indicated by the appearance of small diffraction peaks, after one day under ambient conditions, HEP started to crystallize in the CP product in aqueous suspension (2% hydroxypropyl cellulose). However, no crystallization was observed in the HME product. While crystallization continued in the CP product after four days, only one small diffraction peak was seen with the HME product, suggesting the occurrence of crystallization of HEP. More peaks appeared after seven days, and the peak intensities became stronger in both products. Based on these observations, it is obvious that the HME product has better physical stability than the CP product in suspension. Longer term stability was assessed in a 40° C./75% RH chamber. In the 40° C./75% RH chamber, the two products did not show any sign of crystallization up to three-months. The better physical stability of the HME product is likely due to its less surface area, which causes less penetration of the water molecules into bulk particle and consequently, less plasticizing effect due to the presence of water as well as slower crystallization (Tong and Zografi, 2004)

Both the co-precipitation and the hot melt extrusion processes produced amorphous solid dispersions of HEP which have the following in common: spectroscopic properties, powder X-ray diffraction, true density, and water vapor sorption/desorption behavior. In addition, the API was uniformly dispersed in both products as indicated by the single glass transition temperature in DSC thermograms. However, the co-precipitation process produced solid dispersion with larger specific surface area due to its high porosity and rough particle surface, which provided a faster bulk dissolution compared with the product produced by the hot melt extrusion process. Although both bulk products showed acceptable physical stability for three months in the 40° C./75% RH chamber, the CP product is physically less stable in suspension.

Both the CP and HME products have improved bioavailability over the crystalline form of the drug at doses of 50 mg/kg and 200 mg/kg. Exposures for CP and HME are comparable at low doses, e.g. 50 mg/kg. However, exposures for these two products are significantly different at higher doses, e.g. 250 mg/kg. At the higher dose, HME exhibited a five-fold increase in exposure over the 50 mg/kg dose, while CP exhibited only a two-fold increase.

The poorly soluble compound employed in the present invention can be any compound with aqueous solubility less than 1 mg/mL. The polymeric carrier employed in the hot melt extrusion can include any ionic and nonionic polymer that is suitable for pharmaceutical use, for example, polymethylmethacrylates, polyvinylpyrrolidone, hydroxyethyl cellulose, hydroxypropyl cellulose, hydroxypropyl methylcellulose (hypromellose), ethylcellulose, polyvinypyrrolidone-polyvinylalcohol, hydroxypropyl methylcellulose acetate succicnate (HPMC-AS), hydroxypropyl methylcellulose phthalate, cellulose acetate phthalate, hydroxypropyl cellulose acetate phthalate, methylcellulose acetate phthalate and polymeric surfactants such as poloxamers. The loading of compound in polymer is between 1% and 80% by weight.

### Example 1

### Preparation of Co-Precipitated Solid Dispersion

A solution of HEP (40%) and HPMC-AS (LF grade, 60%) was prepared in acetone. The acetone solution was dropped into acidified water maintained at 2-8° C. to co-precipitate the drug/polymer mixture. The precipitate was then separated by filtration and washed by the acidified water, followed by drying. The dried powder was screened through 40 mesh screen to obtain uniform size particles.

### Example 2

### Preparation of Hot Melt Extruded Solid Dispersion

The 40:60 (by weight) mixture of HEP and HPMC-AS was prepared by mixing in a bin blender (Bohle). The powder mixture was then fed through the hot melt extruder (American Leistritz Corp. 18 mm extruder) with the heating barrels being set at 70-140° C. to obtain extrudate rods. The extrudate rods were cooled to room temperature and milled by mechanical milling methods. The milled granules were passed through 40 mesh screen to obtain uniform particle size distribution.

### Example 3

### Preparation of Spray-Dried Solid Dispersion

HEP (40%) and HPMC-AS (60%) were dissolved in acetone (a common solvent with a low boiling point for both the drug and the polymer). By means of spray drying, the solvent was evaporated, leaving the precipitated drug and polymer. The powder was screened through 40 mesh screen to obtain uniform particle size distribution prior to further evaluation.

Once the solid dispersion is prepared through appropriate approach(s), pharmaceutical formulations, such as capsules and tablets, can be prepared using additional processing techniques commonly known to the person of ordinary skill in the art. The pharmaceutical formulations can be administered to a subject by any route suitable for achieving the desired therapeutic results. However, for this evaluation, the solid dispersions were suspended in an aqueous vehicle for ease of dosing.

### Example 4

X-Ray Diffraction

Reference formulation was prepared by particle size reduction using bead mill to yield particle size in the range of 200-500 nm.

An Advanced Diffraction System (Scintag Inc., Cupertino, Calif., United States) was used to collect powder X-ray diffraction (PXRD) spectra with a CuKα source. The scan was from 2°-40° 2θ with a step size of 0.02° and the residence time of 1.2 seconds with the voltage at 45 kV and the current at 40 mA. Before data collection, the sample was filled into the cavity of the sample holder and the powder surface was leveled. Then the sample holder was loaded onto the 12-position sample changer and PXRD diffraction patters were collected with the instrument set under the above conditions.

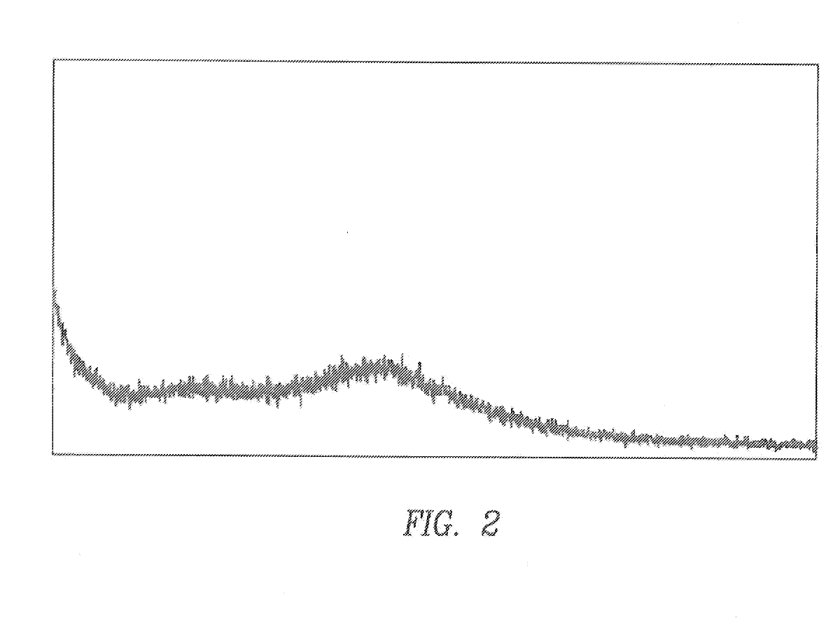

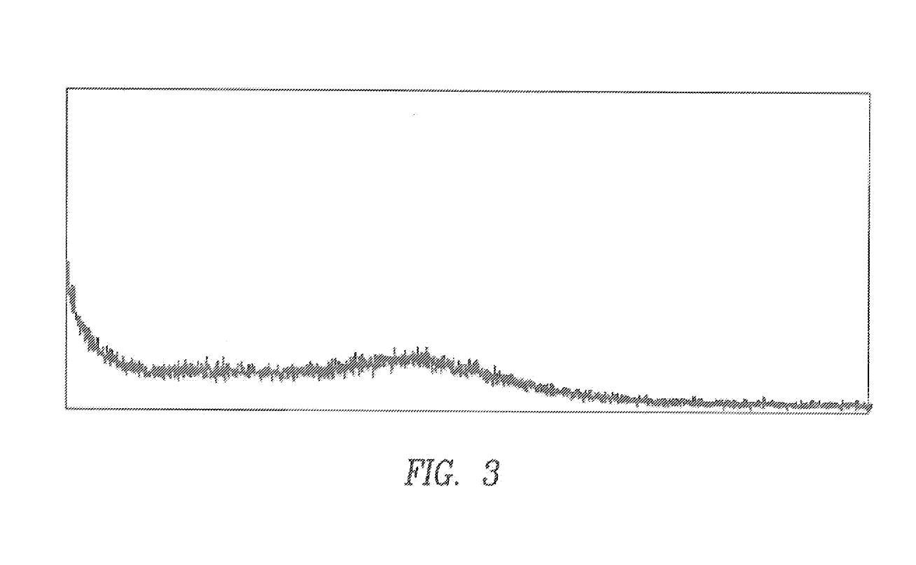

The formulations prepared according to Examples 1 and 2 were shown to be amorphous, as indicated by their powder X-ray diffraction patterns (FIGS. 2 and 3); however, the product of Example 3 was found to be crystalline.

### Example 5

### Glass Transition Temperature

Differential scanning calorimetry (DSC 7, Perkin-Elmer Inc., Wellesley, Mass., United States) was used for measuring glass transition temperature with a nitrogen purge at 30 ml/min and a heating rate at 10° C./min. Hermetic pans carrying a pin hole were used with sample weight around 5 mg. The sample was weighed out in the DSC hermetic pan bottom piece, and then it was sealed with the lid on. After the pan was loaded in the DSC cell, the heating ramp was started from room temperature to 160° C. After sample was run in DSC, the data was analyzed by the Perkin Elmer software to determine the glass transition temperature. Both products have similar glass transition temperatures; 106° C. and 104° C. for the co-precipitate and HME, respectively.

### Example 6

### Specific Surface Area

A TriStar 3000 surface area analyzer (Micromeritics Instrument Corporation, Norcross, Ga., United States) was used to measure the specific surface area by the multiple-point BET method using nitrogen gas as the adsorbate. The samples were vacuum degassed in the tube before analysis where the sample weight was calculated by subtracting the weight of the tube from the total weight (tube+sample) after degassing. The sample tubes were then loaded on the analysis port of the instrument. After evacuation and helium gas purge at liquid nitrogen temperature, the free space volume in the sample tube was measured. The sample tube was then evaluated a second time and thereafter the nitrogen gas adsorption isotherm was determined at specified relative pressures. The amount of gas adsorbed on the sample surface was measured by the desorption of gas. Using the BET equation, the specific surface area was calculated from the nitrogen gas adsorption amounts at their respective relative pressure. The specific surface area was determined as 6.29 m²/g for the co-precipitate and 0.13 m²/g for the HME.

### Example 7

### True Density

AccuPyc 1330 pycnometer (Micromeritics Instrument Corporation, Norcross, Ga., United States) was utilized to measure the true density and nitrogen gas. The true density was calculated from sample weight being divided by its volume. Sample weight was measured by analytical balance. To accurately measure the sample volume, the instrument has two chambers, the sample chamber (volume: Vₛ) and the expansion chamber (volume: Vₓ). The analysis involved an initial purge stage to remove atmospheric gas and replace with pure nitrogen gas. Next, gas filled the sample chamber, equilibrated to a steady state pressure and then the pressure (P₁) was recorded. The gas was then allowed to expand into the expansion chamber where the gas was again allowed to equilibrate and the pressure was recorded (P₂). The gas was then vented to the atmosphere and the cycle is repeated until consecutive measurements are consistent and reproducible. The sample volume was calculated as (Vₛ−Vₓ)/(1−P₂/P₁). True density was determined as 1.33 g/cc and 1.30 g/cc for the co-precipitate and HME products, respectively.

### Example 8

### Dissolution Rate

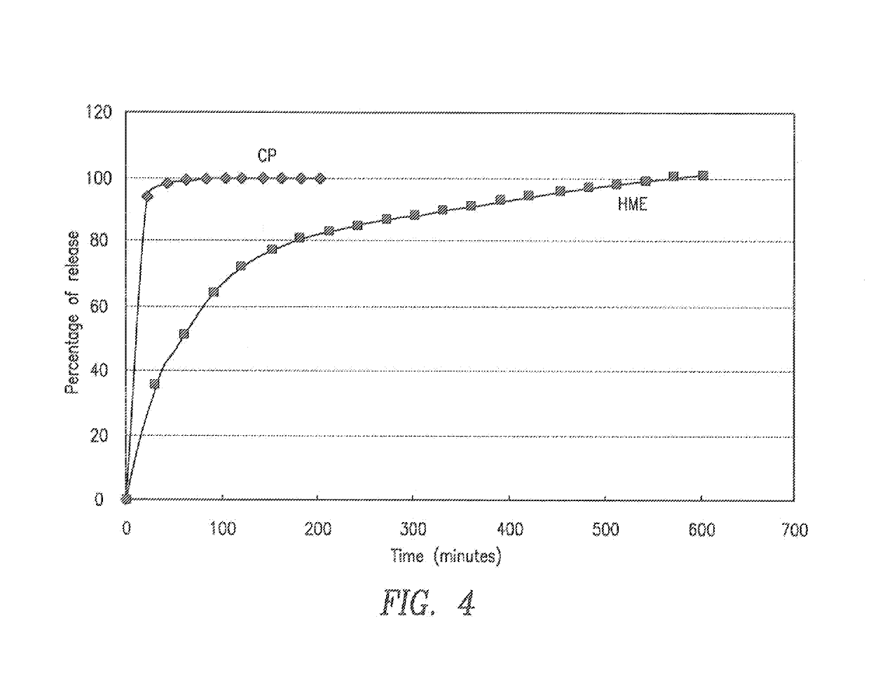

A Distek dissolution apparatus (Distek Dissolution System 2100A, Distek Inc., North Brunswick, N.J., United States) was used to determine dissolution of the CP and HME products in 500 mL of 1% sodium lauryl sulfate (SLS) 50 mM phosphate buffer (pH 6.8) at 37° C. with a stirring rate of 50 rpm. For dissolution test, 100 mg of the CP or HME product was suspended in 1 ml aqueous vehicle (2% hydroxypropyl cellulose in water) and then transferred to the dissolution media for measurement. Due to the large specific surface area, the co-precipitate has a much faster dissolution rate than the HME (FIG. 4).

### Example 9

### Intrinsic Dissolution Rate

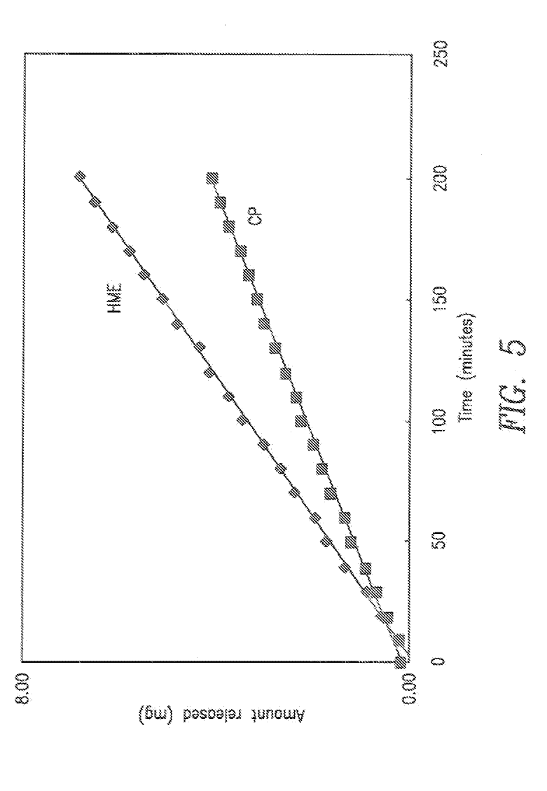

The intrinsic dissolution rate (IDR) was measured using constant surface area pellets in Distek dissolution apparatus (Distek Dissolution System 2100A, Distek Inc., North Brunswick, N.J., United States) paddle method. The powder was compacted into pellets under 2000 pounds using a Carver press (Carver, Inc., Wabash, Ind., United States) for the experiment with a dissolution surface area of 0.5 cm². The pellets were transferred to 500 mL of 1% sodium lauryl sulfate (SLS) 50 mM phosphate buffer (pH 6.8) at 37° C. with a stirring rate of 50 rpm. After experiments, the pellet surface was examined by PXRD and polarized microscopy (Leitz Aristomet, Leitz, Germany). The HME has a higher intrinsic dissolution rate than that of the co-precipitate (FIG. 5).

### Example 10

### Hygroscopicity

A water vapor sorption analyzer (model SGA-100, VTI Corporation, Hialeah, Fla., United States) was employed to assess the hygroscopicity of both products at 25° C. with a sample size of around 15 mg. The experiments were performed under a relative humidity (RH) cycle of 10%→90%→10% at the step of 10%. The equilibrium criterion was set at 0.01% weight change in two minutes or maximum 300 minutes equilibrium time.

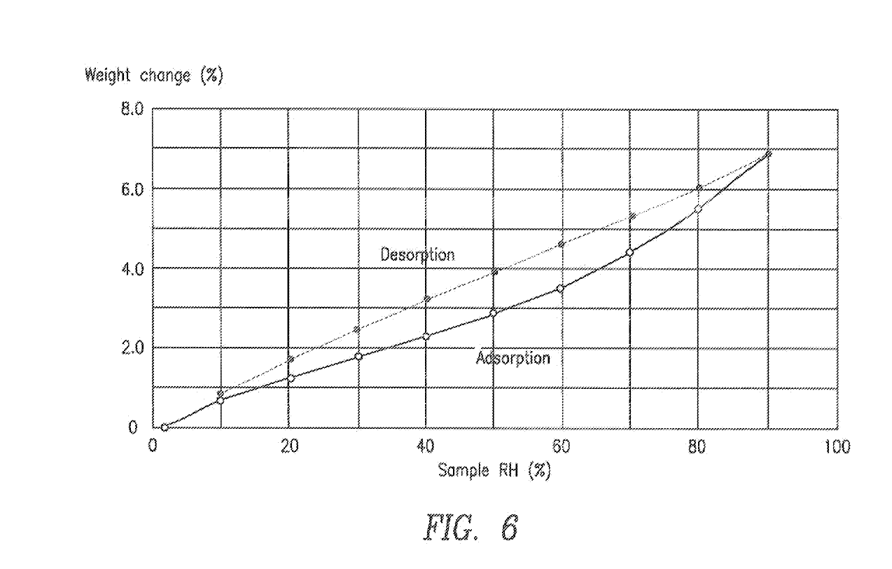

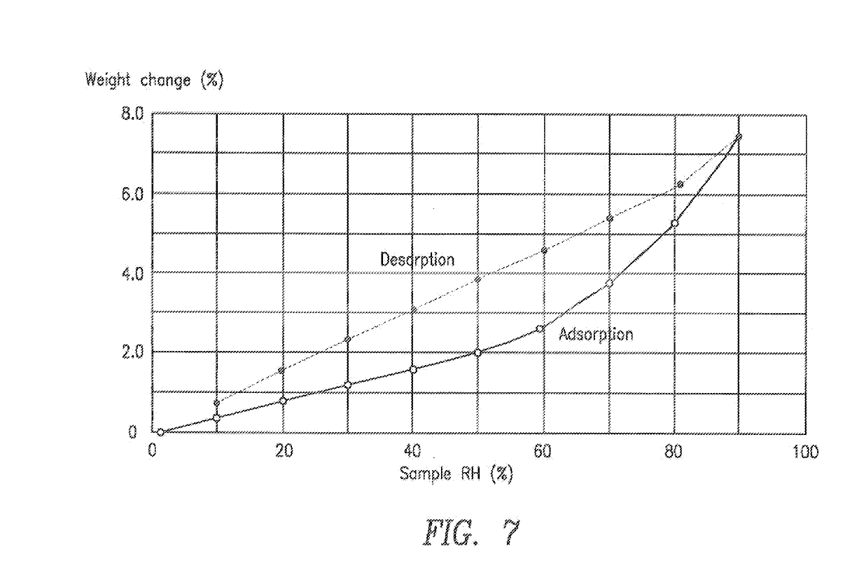

In water vapor sorption/desorption experiments, the two products showed similar hygroscopicity (FIGS. 6 and 7). The comparison of various physico-chemical testing is summarized in Table 1 for the amorphous products produced by Examples 1 and 2.

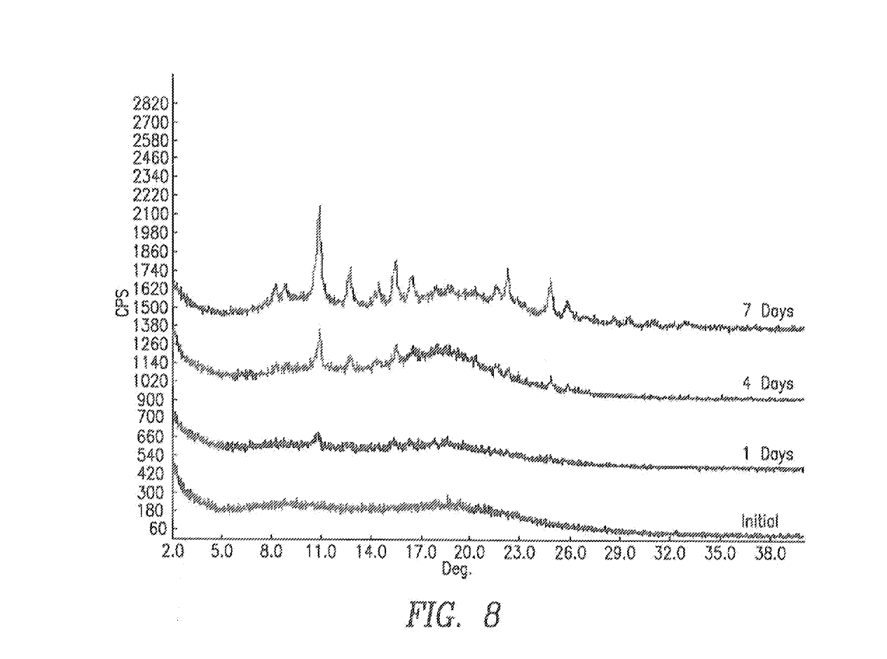

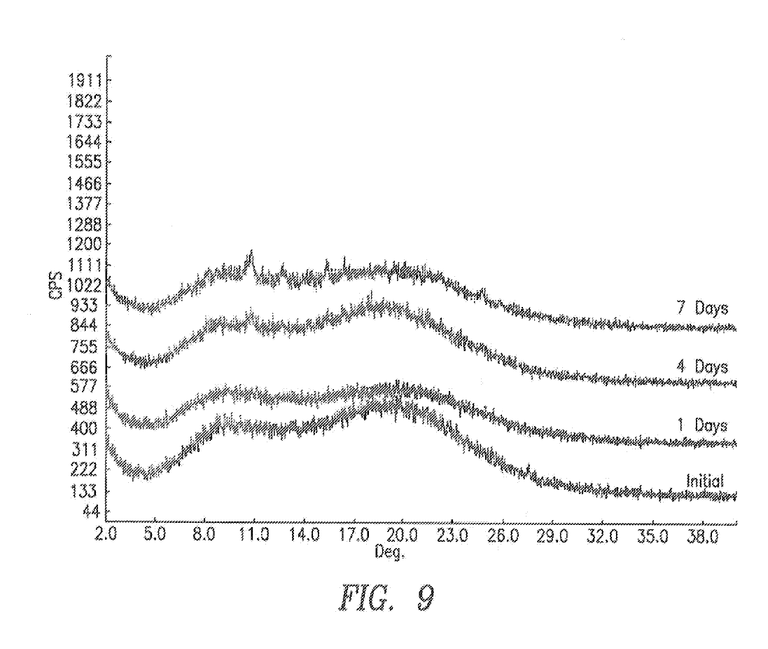

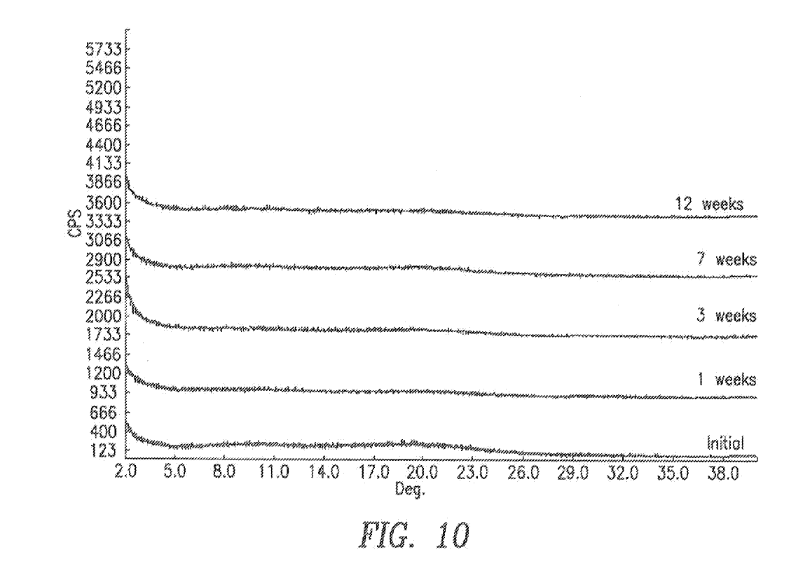

TABLE 1
Physico-chemical properties of Co-precipitate and
Hot melt extrusion solid dispersions.

| Property | Co-precipitate | HME | Comment/ref |
|---|---|---|---|
| Crystallinity by Powder x-ray diffraction | Amorphous | Amorphous | FIGS. 2 and 3 |
| Glass transition temp by DSC (° C.) | 106 | 104 | |
| Specific surface area (m²/g) | 6.29 | 0.13 | |
| True density (g/cc) | 1.33 | 1.30 | |
| Intrinsic dissolution rate (mg/cm²/minute) | 0.0404 | 0.0696 | FIG. 5 |
| Hygroscopicity | Medium | Medium | FIGS. 6 and 7 |
| Physical Stability (suspension in aqueous | Observed | Maintains | FIGS. 8 and 9 |
| vehicle for a week) | conversion to | amorphous | |
| | crystalline | form | |
| Physical stability (storage in 40° C./75% | Good | Good | FIGS. 10 and |
| RH for 3 months) | | | 11 |

The large surface area and faster dissolution rate suggests rapid onset of action for the CP product. On the other hand, the slower dissolution rate suggests sustained release profile for the HME product. Despite the slower dissolution rate from the bulk dissolution, the intrinsic dissolution rate for HME is higher suggesting a constant release of drug with good stability of the amorphous form. Due to its less hygroscopicity, HME is predicted to be more stable than CP.

### Example 11

### Physical Stability

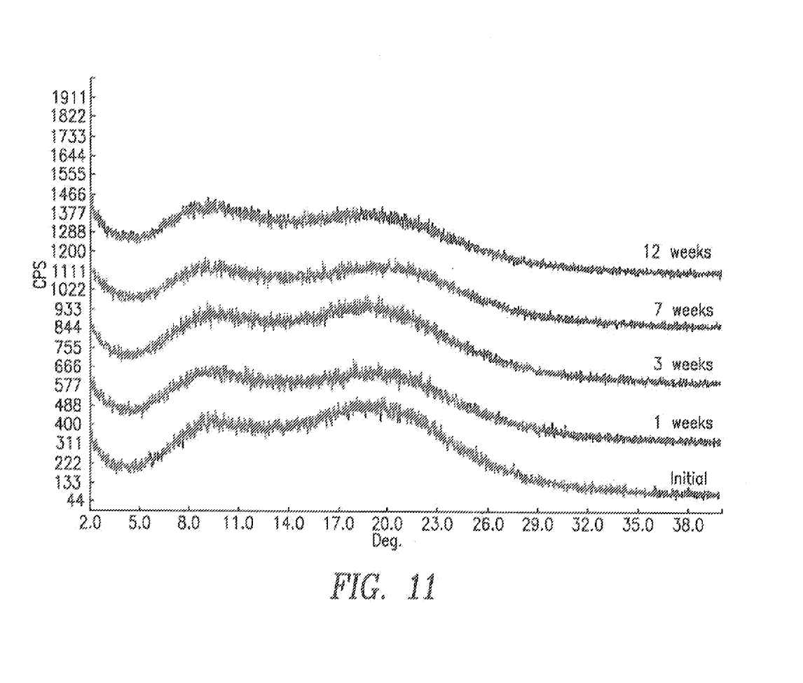

The stability of both products was evaluated in aqueous suspension and in 40° C./75% RH chamber. Indeed, after the two products were suspended in aqueous vehicle for a week, HME showed a much slower crystallization rate (FIGS. 8 and 9), likely due to the much slower penetration of water molecules into HME particles. However, after storage in 40° C./75% RH chamber for three months, there was no crystallization detected in either of the products by powder X-ray diffraction (FIGS. 10 and 11), suggesting both products are physically stable for at least three months under this storage condition. The superior suspension stability of HME clearly indicates the advantage of the hot melt extrusion process to produce stable amorphous solid dispersion.

Manufacture of an amorphous formulation has been a challenging task, especially its scale-up. From this perspective, the hot melt extrusion process is much more robust due to continuous processing and the availability of equipment from R & D to commercial scale. In contrast, the co-precipitation method depends on the solubility of drug and polymer in the common solvent, and on the challenges associated with controlled precipitation and the scale-up of batch mode processing.

### Example 12

### In Vivo Testing

The data summarized in Table 2 shows the exposure of HEP after rats were dosed with the CP and HME products. The results indicate that, compared with crystalline drug suspension (nanoparticle size range), both products have much improved bioavailability, and furthermore, the HME product has superior dose-exposure proportionality than the CP product at doses of 50 mg/kg and 250 mg/kg.

At the dose level of 50 mg/kg, the data (Table 2) show that the exposure of the solid dispersion formulations (CP and HME products) was ˜40 folds higher compared with the nano-formulation (crystalline). Further increase in the dose showed no improvement in the exposure for the nano-formulation. Although the exposure of CP and HME products was comparable at 50 mg/kg dose, significant differences were observed at the higher dose level, i.e. 250 mg/kg. HME showed dose dependent increase (5 folds) in exposure over the 50 mg/kg dose; however, CP showed only a 2 folds increase. The superior performance of HME can be explained based on the differences in the solid state properties, such as low surface area, high bulk density and slightly lower hygroscopicity. However, the superior pharmacokinetic performance and stability could not be predicted especially with fast intrinsic dissolution rate for the HME product.

TABLE 2
Rat PK results of CP vs. HME formulation of Drug A compared with its nano formulation.

| | | Nano | CP | CP | HME | HME |
|---|---|---|---|---|---|---|
| | | formulation | 50 | 250 | 50 | 250 |
| Parameter | Units | mg/kg | mg/kg | mg/kg | mg/kg | mg/kg |
| Dose | Mg/kg | 50 | 50 | 250 | 50 | 250 |
| AUC | Ng*Hours/mL | 12,092 | 505,506 | 987,900 | 468,415 | 1,795,540 |
| Cₘₐₓ | ng/mL | 1046 | 980,333 | 151,667 | 76,900 | 157,000 |
| Tₘₐₓ | Hours | 5.5 | 1.33 | 1.5 | 2 | 2.66667 |
| AUC/Dose | ng*Hours/mL/mg/kg | 46 | 10110 | 3952 | 9368 | 7182 |
| Cₘₐₓ/Dose | ng/mL/mg/kg | 4.2 | 1961 | 607 | 1538 | 628 |

1. A physically stable solid dispersion comprising (2S,3S)-2-{(R)-4-[4-(2-hydroxy-ethoxy)-phenyl]-2,5-dioxo-imidazolidin-1-yl}-3-phenyl-N-(4-propionyl-thiazol-2-yl)-butyramide (HEP) and an ionic or nonionic polymer.

2. The solid dispersion of claim 1, wherein the ionic or nonionic polymer is selected from the group consisting of polymethylmethacrylates, polyvinylpyrrolidone, hydroxyethyl cellulose, hydroxypropyl cellulose, hydroxypropyl methylcellulose (hypromellose), ethylcellulose, polyvinypyrrolidone-polyvinylalcohol, hydroxypropyl methylcellulose (hypromellose) acetate succinate (HPMC-AS), hydroxypropyl methylcellulose phthalate, cellulose acetate phthalate, hydroxypropyl cellulose acetate phthalate, methylcellulose acetate phthalate and poloxamers.

3. The solid dispersion of claim 1, wherein the ionic or nonionic polymer is hydroxypropylmethyl cellulose acetate succinate.

4. The solid dispersion of claim 1 wherein the amount of HEP in the ionic or nonionic polymer is between 1% and 80% by weight.

5. The solid dispersion of claim 1 which is prepared by hot melt extrusion.

6. The solid dispersion of claim 1 which is prepared by co-precipitation.

7. A solid dispersion comprising (2S,3S)-2-{(R)-4-[4-(2-hydroxy-ethoxy)-phenyl]-2,5-dioxo-imidazolidin-1-yl}-3-phenyl-N-(4-propionyl-thiazol-2-yl)-butyramide (HEP) and an ionic or nonionic polymer which has a higher bioavailability than the crystalline form of the HEP.

8. A solid dispersion comprising (2S,3S)-2-{(R)-4-[4-(2-hydroxy-ethoxy)-phenyl]-2,5-dioxo-imidazolidin-1-yl}-3-phenyl-N-(4-propionyl-thiazol-2-yl)-butyramide (HEP) and an ionic or nonionic polymer wherein the HEP exists in an amorphous form.

9. A method for preparing a solid dispersion of (2S,3S)-2-{(R)-4-[4-(2-hydroxy-ethoxy)-phenyl]-2,5-dioxo-imidazolidin-1-yl}-3-phenyl-N-(4-propionyl-thiazol-2-yl)-butyramide (HEP) and an ionic or nonionic polymer which comprises forming a powdered mixture of the HEP and the polymer and extruding the mixture through a hot melt extruder.
